(19) United States
(12) Patent Application Publication
(10) Pub. No.: US 2020/0124568 A1
(43) Pub. Date: Apr. 23, 2020

(54) APPARATUS AND METHOD FOR DETERMINING ANTIBIOTIC RESISTANCE USING MALDI-TOF MASS SPECTROMETRY

(71) Applicant: NOSQUEST CO., LTD., Seongnam-Si (KR)

(72) Inventors: Hyung Soon Park, Seoul (KR); Seong Jin Ahn, Seoul (KR); Kyoohyoung Rho, Seoul (KR); Eung Joon Jo, Seongnam-Si (KR)

(73) Assignee: NOSQUEST CO., LTD., Seongnam-Si (KR)

(21) Appl. No.: 16/624,596

(22) PCT Filed: Nov. 13, 2018
(86) PCT No.: PCT/KR2018/013807

§ 371 Date: Dec. 19, 2019

(30) Foreign Application Priority Data
Nov. 14, 2017 (KR) 10-2017-0151780

(51) Int. Cl. G01N 27/64; H01J 49/16; H01J 49/40; H01J 49/00; C12Q 1/18; G16B 40/00

(52) U.S. Cl. CPC G01N 27/64; H01J 49/164; G16B 40/00; H01J 49/0036; C12Q 1/18; H01J 49/40

(57) ABSTRACT

The present invention relates to an apparatus for determining resistance of microorganisms, which may comprise: a storage unit including a database for storing first region mass spectrum data for a plurality of microorganisms and second region mass spectrum data used to determine resistance of each of the plurality of microorganisms; and a processor including a resistance determination module which determines resistance to an antibiotic by comparing the second region mass spectrum data with mass spectrum data of a microorganism sample to be determined.

### TECHNICAL FIELD

Example embodiments relate to a method and apparatus for determining an antibiotic resistance of a microorganism and, more particularly, to a method and apparatus for performing a resistance determination and an identification on a microorganism based on a matrix assisted laser desorption/ionization time-of-flight mass spectrometry (MALDI-TOF MS) for a microorganism sample.

### BACKGROUND ART

A microorganism test is increasingly required for the health and hygiene of the public in various fields such as agricultural, fisheries, and animal husbandry fields, sanitation facility inspection for food processing and distribution, and sanitation inspection of soil and water resources as well as a medical field to treat diseases caused by microorganism infection. Also, with developments of microorganism industries that create new values using microorganisms such as fermented foods, organic fertilizers, bioenergy, vaccines/antibiotics, and microorganism environmental preparations, desires for accurate strain cultivation and classification are increasing.

In particular, the microorganism test in the medical field is directly related to a patient's life suffering from congestive diseases such as sepsis. Also, since an appropriate treatment is determined based on a microorganism identification result, promptness and accuracy of an analysis are simultaneously required for a microorganism identification test. A microorganism identification mass spectrometer may significantly reduce an analysis time from several hours or days to several minutes and may classify thousands of microorganism subspecies.

A matrix assisted laser desorption/ionization time-of-flight (MALDI-TOF) based microorganism identification system may construct a protein mass spectrum patterned database for each microorganism based on mass spectrums of proteins varying based on a type of microorganism and identify a microorganism through a comparative analysis.

The MALDI-TOF based microorganism identification system is a low-cost and high-efficiency identification system in comparison to a DNA sequencing-based identification method, and may be an important tool for rapid microorganism identification. In addition, the system may be widely used in terms of application to origin certification of domestic resources, diagnosis of various infectious microorganisms, food industries, and quarantine inspection.

A strain identification process may quickly identify a strain through processing after cultivation of the strain. As a method that can be processed within ten minutes from a strain sample processing to identification after strain cultivation, the method may identify a strain by acquiring MALDI-TOF data on an unknown strain, comparing the acquired data to MALDI-TOF data in a database built in advance, and identifying a matching strain.

In the case of a susceptibility test for screening antibiotics for pathogenic microorganisms and determining resistances, at least 18 hours may be taken after a microorganism is cultured and the accuracy may also be reduced.

Accordingly, there is a desire for a system for identifying a microorganism based on a mass spectrometry and determining whether the microorganism has a resistance to a specific drug.

### DISCLOSURE OF INVENTION

### Technical Solutions

According to an aspect, there is provided an apparatus for determining a resistance of microorganisms, the apparatus including a storage including a database to store first region mass spectrum data associated with a plurality of microorganisms and second region mass spectrum data used for determining a resistance of each of the plurality of microorganisms, and a processor configured to determine a resistance to an antibiotic by inputting mass spectrum data of a microorganism sample to be determined, to a statistic or machine-learning resistance determining module that determines a resistance using the second region mass spectrum data as input data.

The apparatus may further include a mass analyzer configured to acquire the first region mass spectrum data and the second region mass spectrum data based on a matrix assisted laser desorption/ionization time-of-flight mass spectrometry (MALDI-TOF MS) in association with the microorganism sample to be determined.

The processor may be configured to identify a microorganism by inputting mass spectrum data of a microorganism sample to be determined, to a statistic or machine-learning identifying module that identifies a microorganism using the first region mass spectrum data as input data.

According to another aspect, there is also provided an apparatus for determining a resistance of a microorganism, the apparatus including a communicator configured to connect to a cloud server including a database that stores first region mass spectrum data associated with a plurality of microorganisms and second region mass spectrum data used for determining a resistance of each of the plurality of microorganisms, and a processor configured to determine a resistance to an antibiotic by inputting mass spectrum data of a microorganism sample to be determined, to a statistic or machine-learning resistance determining module that determines a resistance using the second region mass spectrum data as input data.

The apparatus may further include a mass analyzer configured to acquire the first region mass spectrum data and the second region mass spectrum data based on a MALDI-TOF MS in association with the microorganism sample to be determined.

The processor may be configured to identify a microorganism by inputting mass spectrum data of a microorganism sample to be determined, to a statistic or machine-learning identifying module that identifies a microorganism using the first region mass spectrum data as input data.

According to another aspect, there is also provided a method of determining a resistance of a microorganism, the method including storing, by a storage, first region mass spectrum data associated with a plurality of microorganisms and second region mass spectrum data used for determining a resistance of each of the plurality of microorganisms in a database, and determining, by a processor, a resistance to an antibiotic by inputting mass spectrum data of a microorganism sample to be determined, to a statistic or machine-learning resistance determining module that determines a resistance using the second region mass spectrum data as input data.

The method may further include acquiring, by a mass analyzer, the first region mass spectrum data and the second region mass spectrum data based on a MALDI-TOF MS in association with the microorganism sample to be determined.

The method may further include identifying, by the processor, a microorganism by inputting mass spectrum data of a microorganism sample to be determined, to a statistic or machine-learning identifying module that identifies a microorganism using the first region mass spectrum data as input data.

According to another aspect, there is also provided an operation method of a microorganism resistance determining apparatus, the method including acquiring, by a mass analyzer, first region mass spectrum data associated with a microorganism sample to be determined, identifying, by a processor, a microorganism by inputting the first region mass spectrum data of the microorganism sample to be determined, to a statistic or machine-learning identifying module that identifies a microorganism using mass spectrum data as input data, acquiring, by the mass analyzer, second region mass spectrum data associated with the microorganism sample, and determining a resistance to an antibiotic by inputting mass spectrum data of the microorganism sample to a statistic or machine-learning resistance determining module that determines a resistance using the first region and second region mass spectrum data as input data.

The operation method may further include storing, by a storage, first region mass spectrum data associated with a plurality of microorganisms and second region mass spectrum data used for determining a resistance of each of the plurality of microorganisms in a database.

The machine-learning resistance determining module may be configured to determine whether a microorganism has a resistance to an antibiotic through a machine learning performed by converting the first region mass spectrum data and the second region mass spectrum data into a feature matrix and applying the feature matrix as an input value.

The operation method may further include calibrating the microorganism sample using a correction material acting on the second region to increase an accuracy of mass spectrum data on the second region.

The operation method may further include interworking, by a communicator, with a cloud server that stores first region mass spectrum data associated with a plurality of microorganisms and second region mass spectrum data used for determining a resistance of each of the plurality of microorganisms, in a database.

According to another aspect, there is also provided a non-transitory computer-readable medium comprising a program for instructing a computer to perform the microorganism resistance determination method or an operation method of a microorganism resistance determining apparatus.

### BRIEF DESCRIPTION OF DRAWINGS

FIG. 1 is a block diagram illustrating a microorganism analyzing apparatus according to an example embodiment.

FIG. 2 is a block diagram illustrating a processor according to an example embodiment.

FIG. 3 is a flowchart illustrating a microorganism analysis method according to an example embodiment.

FIG. 4 illustrates a process of acquiring a microorganism mass analysis signal using a matrix assisted laser desorption/ionization time-of-flight mass spectrometry (MALDI-TOF MS) according to an example embodiment.

FIG. 5 illustrates a MALDI-TOF based microorganism identification and antibiotic resistance determination apparatus according to an example embodiment.

### BEST MODE FOR CARRYING OUT THE INVENTION

Hereinafter, example embodiments will be described in detail with reference to the accompanying drawings. It should be understood, however, that there is no intent to limit this disclosure to the particular example embodiments disclosed. Like reference numerals refer to like elements throughout.

The terms used herein were selected to include current, widely-used, general terms, in consideration of the functions of the present disclosure. However, the terms may represent different meanings according to the intentions of those skilled in the art or according to customary usage, the appearance of new technology, etc. As such, the terms used in the specification are not to be defined simply by the name of the terms but are to be defined based on the meanings of the terms as well as the overall description of the present disclosure.

Additionally, certain terms may have been arbitrarily selected, and in this case, their meanings are described herein. Accordingly, the terms used in this specification should be interpreted on the basis of substantial implications that the terms have and the contents across this specification not the simple names of the terms.

A typical diagnostic technique for a specific target biomarker may have an incompleteness and a variability of a single biomarker. To solve this, an apparatus for identifying a microorganism and determining a drug resistance of the microorganism using a microorganism analysis technology based on a mass spectrum analysis is proposed.

The mass spectrum analysis-based microorganism analysis technology may identify a distinguishable pattern by analyzing biodata (MALDI-TOF MS data) obtained from a sample based on a statistic algorithm, match biodata of a unknown sample and a result stored in a database, and identify a group to which the unknown sample belongs, so as to perform identification. As the biodata increases due to an expansion of the data, the method may obtain an advanced analysis result that ensures higher accuracy and detailed classification.

Accordingly, it is possible to overcome the incompleteness (lack of accuracy and specificity of the diagnosis) and the variability (decrease in usefulness as a biomarker over time) of the typical target-oriented single biomarker scheme and achieve an improved result by a constantly evolving system.

In addition, through the mass spectrum microorganism analysis, various mixed disease markers may be accurately distinguished and detected simultaneously. Also, high sensitivity detection with high detection limits is possible and a detected signal may have less ambiguity.

The proposed database-based microorganism analysis method may provide a database for efficiently managing metabolite a matrix assisted laser desorption/ionization time-of-flight (MALDI-TOF) data including protein data and lipid data of microorganisms and provide software for determining identification and a resistance of a microorganism by comprehensively analyzing a MALDI-TOF pattern to achieve a high speed and accuracy of the microorganism analysis.

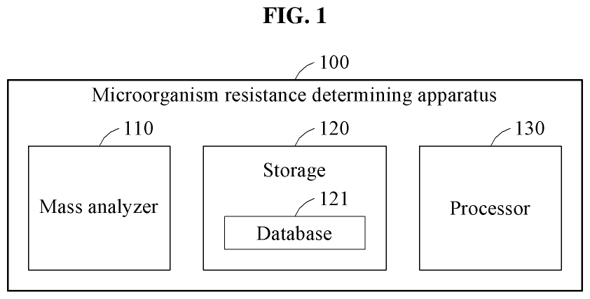

FIG. 1 is a block diagram illustrating a microorganism resistance determining apparatus according to an example embodiment. A microorganism resistance determining apparatus 100 may include a mass analyzer 110, a storage 120, and a processor 130.

The microorganism resistance determining apparatus 100 may analyze a mass spectrum of a microorganism sample, identify a microorganism included in the sample, and determine whether the microorganism has a resistance to an antibiotic.

To analyze the mass spectrum of the microorganism sample to be determined by the microorganism resistance determining apparatus 100, a pretreatment of the microorganism sample may be performed. The pretreatment of the microorganism sample may be performed manually by a user who performs the analysis or performed using a separate microorganism sample pretreatment device.

The sample pretreatment device may perform the pretreatment by applying a specific solution to the microorganism sample, for example, but not limited thereto.

The mass analyzer 110 may acquire mass spectrum data of the microorganism sample. The mass analyzer 110 may analyze the mass spectrum using the pretreated microorganism sample. The mass analyzer 110 may use, for example, but not limited to, a MALDI-TOF mass spectrum analysis method to acquire the mass spectrum data of the sample.

Specifically, the mass analyzer 110 may be a MALDI-TOF mass analyzing device and perform a mass analysis on an input microorganism sample. However, this is merely an example and various mass spectrum analyzing methods and apparatuses are applicable.

The storage 120 may include a database 121 that stores the mass spectrum data and a model obtained by processing the data. Specifically, the storage 120 may include the database 121 that stores a first region mass spectrum data of the plurality of microorganisms and a statistic model or machine-learning model obtained by processing mass spectrum data of one or more regions added to determine a resistance of each of the plurality of microorganisms. The region added to determine the resistance may differ based on a characteristic of each of the microorganisms.

The storage 120 may be operated as an external cloud server. For example, the microorganism resistance determining apparatus 100 may further include a communicator (not shown) interworking with the external cloud server. In this example, the database 121 may be stored in the external cloud server.

The first region mass spectrum data of the plurality of microorganisms stored in the database 121 may be used by the processor 130 to identify the microorganism. Also, second region mass spectrum data may be used by the processor to determine a resistance of the microorganism along with the first region mass spectrum data.

The first region mass spectrum data may be, for example, but not be limited to mass spectrum data of a region of a molecular weight between 2000 and 20000 used for microorganism identification. This is merely an example and includes an area varied by those skilled in the art.

The second region mass spectrum data may be mass spectrum data on a molecular weight region that is specialized for determining a resistance of each microorganism. For example, the second region mass spectrum data may be high-resolution mass spectrum data on a region of a molecular weight between 1000 and 4000 for determining whether a first microorganism has a resistance to a predetermined antibiotic.

This is merely an example, and may be mass spectrum data on another molecular weight region that is specialized for each microorganism of another region. Thus, to determine whether a second microorganism has a resistance to a predetermined antibiotic, high-resolution mass spectrum data on a region of a molecular weight between 6000 and 10000 may be stored.

The storage 120 may include the database 121 that stores information associated with a mass spectrum of a microorganism.

The database 121 may store mass spectrum data on an entire region to be acquired by performing a mass spectrum analysis on various microorganisms. Also, in a case of a microorganism for which whether the microorganism has a resistance to an antibiotic is to be determined among the microorganism, a statistic model or machine-learning model obtained by processing mass spectrum data on a specialized region together may be stored.

For example, in association with the first microorganism, mass spectrum data on a region of the molecular weight between 2000 and 20000 as the entire region (or a first region) may be stored. Also, when the first microorganism has the resistance to the antibiotic, a statistic model or machine-learning model obtained by processing high-resolution mass spectrum data on a region of the molecular weight between 1000 and 4000 as a second region together may be stored.

A range of the second region specialized for determining the resistance to the antibiotic may vary based on a type of a microorganism. Also, a third region and a fourth region may be additionally used to perform a resistance analysis of a microorganism. Hereinafter, mass spectrum analysis data on the second region will be described based on a classification into a high MW (molecular weight), a middle MW (molecular weight), and a low MW (molecular weight).

For example, a material corresponding to a mass range of at least a molecular weight of 20,000 or at least a predetermined molecular weight may be classified as a high MW substance region. In this example, mass spectrum data in the corresponding region may be defined as mass spectrum data of a high-molecular weight region. Also, the database 121 may store data corrected through a calibration performed using a high-molecular weight material mixture. The high MW material mixture may be, for example, a protein mixture or a polymer mixture.

The mass spectrum information on the high-molecular weight material is merely described as an example, and the suggested molecular weight range may also be changed by those skilled in the art.

A middle-molecular weight region may refer to a region corresponding to a mass range of the molecular weight between 2,000 and 20,000 or a predetermined molecular weight less than 100,000. Thus, mass spectrum data on the middle-molecular weight region may be mass spectrum information on a material having the molecular weight of the exemplary range.

The mass spectrum information on the middle-molecular weight region may be corrected through a calibration performed using a middle-molecular weight material mixture. The middle-molecular weight material may be, for example, a peptide and protein mixture or a middle-molecular weight polymer mixture.

A low-molecular weight region may refer to a region corresponding to a mass range of a molecular weight of 3,000 or at most a predetermined molecular weight. Also, a calibration may be performed using other low-molecular weight material mixtures.

For example, with respect to a specific microorganism, a method of identifying the microorganism using mass spectrum data of the first region and determining whether the microorganism has a resistance based on mass spectrum data of the second region, the third region, and the fourth region in addition to the mass spectrum data of the first region is also possible.

The processor 130 may determine the resistance to the antibiotic based on a result obtained by applying mass spectrum data of the microorganism sample to be determined, as an input value of the statistic model or machine-learning model stored in the database 121.

That is, the processor 130 may identify the microorganism using the mass spectrum data on the microorganism sample to be determined, and then determine whether the microorganism has a resistance to a specific antibiotic.

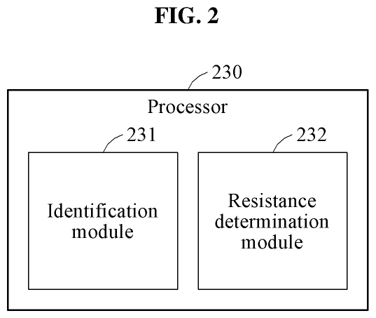

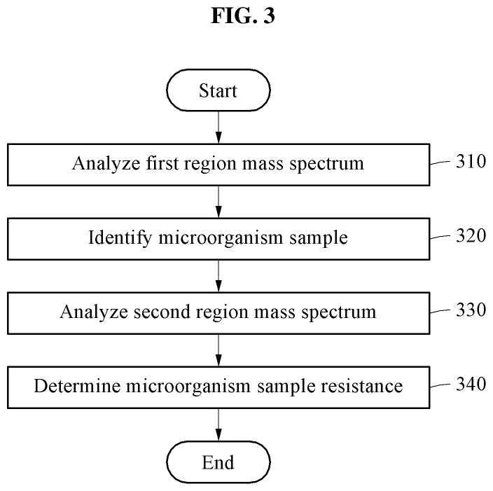

The resistance determiner 130 may sequentially perform identification and resistance determination of the microorganism sample. For example, the mass analyzer 110 may acquire the first region mass spectrum data of the microorganism sample, compare the acquired data to the database, and identify a matching microorganism. In a case of a microorganism requiring the resistance determination, the mass analyzer 110 may perform calibration on the second region specialized for the corresponding microorganism, thereby acquiring corrected mass spectrum data on the second region. Also, the mass analyzer 110 may determine whether the microorganism sample has the resistance based on a result obtained by applying the first and second mass spectrum data as an input value of a statistic model or machine-learning model stored in the database. The calibration for the second region may be performed using a correction material including a protein and peptide mixture suitable for a specific region such that mass spectrum data of the specific region specific to the identified microorganism is precisely obtained. A configuration of the processor 130 identifying a microorganism and determining whether the microorganism has a resistance will be described in detail with reference to FIG. 2, and an operation thereof will be described in detail with reference to FIG. 3.

FIG. 2 is a block diagram illustrating a processor according to an example embodiment. A processor 230 may correspond to the processor 130 of FIG. 1. The processor 230 may include an identification module 231 and a resistance determination module 232.

The processor 230 may compare mass spectrum data of various microorganisms stored in a database to mass spectrum data of a microorganism sample to be determined, thereby identifying a microorganism or determining a resistance to an antibiotic. Specifically, the identification module 231 of the processor 220 may identify the microorganism sample by comparing the mass spectrum data of the microorganism sample to first region mass spectrum data of the database. Meanwhile, the resistance determination module 232 may determine a resistance of the microorganism sample based on a result obtained by applying first and second region mass spectrum data of the microorganism to a statistic model or machine-learning model made by processing the mass spectrum data of the microorganism sample to be determined, as an input.

FIG. 3 is a flowchart illustrating a microorganism analysis method according to an example embodiment. A microorganism analysis method performed by a microorganism analyzing apparatus may include first region mass spectrum analyzing operation 310, microorganism sample identifying operation 320, second region mass spectrum analyzing operation 330, and microorganism sample resistance determining operation 340.

In the microorganism analyzing method, an operation of pretreating a sample using a separate microorganism sample pretreatment device may be performed. In the operation of pretreating the microorganism sample, a pretreatment may be performed on a microorganism sample to be analyzed. Specifically, the pretreatment process may be performed by culturing a microorganism, dropping, on a plate, an extracted sample from which the microorganism is removed for mass spectrum measurement, mixing the sample with a matrix solution, and drying the mixture.

The first region mass spectrum analyzing operation 310 may be an operation of acquiring, by a mass analyzer, mass spectrum data on a first region using a method such as MALDI-TOF. Specifically, the first region may be an entire region in which the mass spectrum data is acquired. For example, as the entire region including a macromolecule, a middle molecule, and a small molecule, mass spectrum data on a region of a molecular weight between 2000 and 20000 may be acquired.

The microorganism sample identifying operation 320 may be an operation of performing, by an identification module of a processor, microorganism identification by comparing the acquired mass spectrum data to mass spectrum data on a microorganism sample stored in a database.

The second region mass spectrum analyzing operation 330 may be an operation of analyzing second region mass spectrum corrected by performing a calibration on a second region when whether the microorganism has a resistance to an antibiotic is required to be determined based on an identification result of the microorganism sample to be determined.

More specifically, in this operation, the mass analyzer may acquire mass spectrum data on the second region specialized to determine a resistance of the microorganism. The second region may be a molecular weight region and set to be a range differing for each microorganism. For example, in a case of a first microorganism, a high-molecular weight region may be the second region. Also, in a case of a second microorganism, a low-molecular weight region may be the second region. That is, high-resolution mass spectrum data on a region specialized to determine whether to have a resistance for each microorganism may be acquired.

The microorganism sample resistance determining operation 340 may be an operation of determining, by a resistance determination module of the processor, whether the microorganism has a resistance. Specifically, whether the microorganism has a resistance to a specific antibiotic or an antibiotic to which the microorganism has a resistance may be determined based on a result obtained by applying the acquired mass spectrum data to a statistic model or machine-learning model stored in a database as an input value.

Resistance determination may be performed by the resistance determination module of the processor. The resistance determination module may determine whether the microorganism has a resistance to an antibiotic based on a result obtained by applying first region mass spectrum data and second region mass spectrum data stored in the database, as an input value of a statistic model or machine-learning model obtained by processing the mass spectrum data of the microorganism sample to be determined.

For example, with respect to various strains of the first microorganism that are resistant or susceptible to a specific antibiotic, mass spectrum data of a first region of a molecular weight between 2000 and 20000 and mass spectrum data of a second region of a molecular weight between 1000 and 4000 may be acquired. The acquired mass spectrum data may be processed into a feature matrix through an appropriate processing process. Such an appropriate processing process may include, for example, spectrum quality management, smoothing, baseline correction, intensity calibration, peak detection, and peak selection using a signal to noise ratio (SNR). Using the feature matrix as a model data set, a random forest model may be trained through 4-fold cross validation repeated 20 times. When an unknown microorganism is identified as the first microorganism, mass spectrums of the first region of the molecular weight between 2000 and 20000 and the second region of the molecular weight between 1000 and 4000 may be acquired. A feature vector obtained through the above processing process may be applied as an input value of a trained machine-learning model, so that whether the microorganism has the resistance to the antibiotic and a probability value may be obtained.

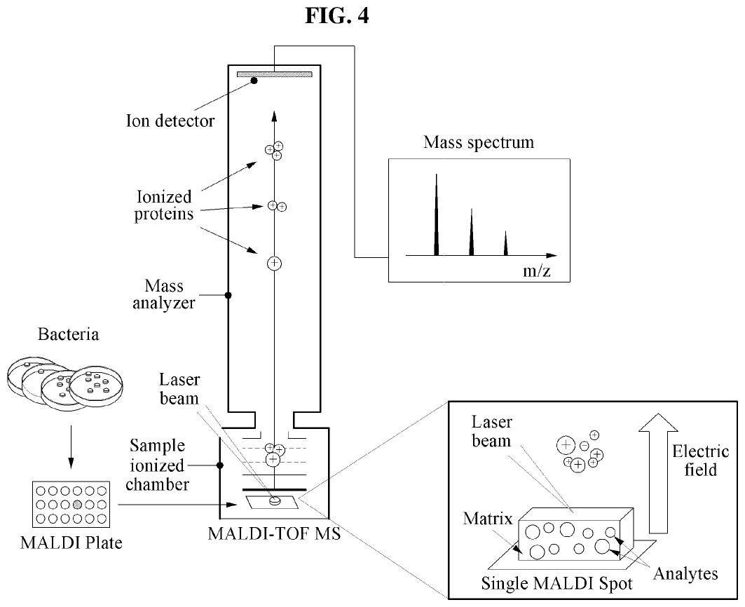

FIG. 4 illustrates a process of acquiring a microorganism mass analysis signal using a MALDI-TOF MS according to an example embodiment.

Describing sequentially from the left, a microorganism sample may be disposed on a MALDI Plate to be cultured. The sample may require a separate pretreatment process. In a sample ionized chamber, the microorganism sample may be ionized using a laser beam. The ionized sample may be detected by an ion detector located on one side of a mass analyzer, and a mass spectrum may be acquired.

In the ionization using the laser beam, the sample to be analyzed may be located in a matrix solution and ionized by applying the laser beam. In this instance, an electric field may be applied such that an ion reaches the ion detector.

The mass spectrum may be detected using the ion reaching the ion detector.

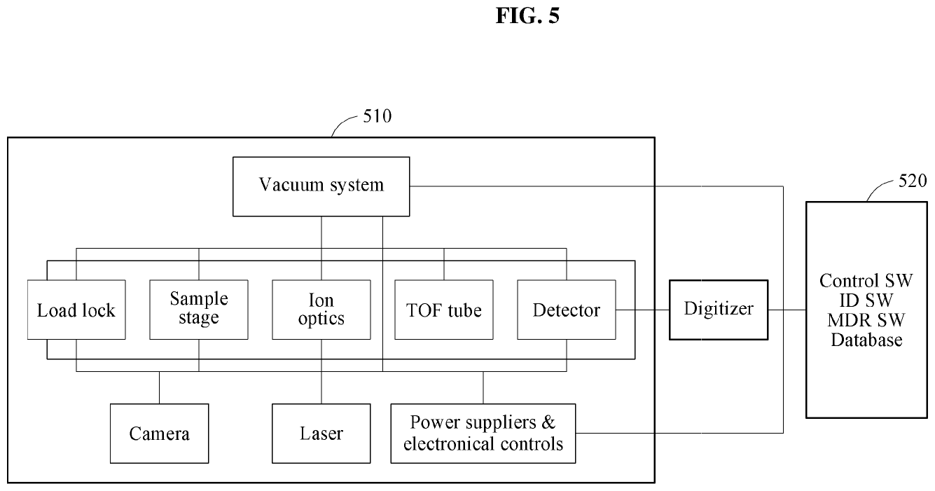

FIG. 5 illustrates a MALDI-TOF-based microorganism identification and antibiotic resistance determination apparatus according to an example embodiment. A MALDI-TOF-based microorganism identification and antibiotic resistance determination apparatus may include a MALDI-TOF mass spectrometer 510 and a microorganism resistance determining device 520.

The MALDI-TOF mass spectrometer 510 may include a load lock, a sample stage, ion optics, a TOF tube, and a detector, under a vacuum system. In addition, a camera, a laser, a power supply, and an electronic controller may also be included.

When a digitizer digitizes mass spectrum data acquired by MALDI-TOF mass spectrometer 510, the microorganism resistance determining device 520 may analyze the mass spectrum data. The microorganism resistance determination apparatus may include Control SW, ID SW, MDR SW, and a database.

The microorganism sample may be identified by the ID SW. When a mass spectrum of a specific region is acquired again for the identified microorganism, the MDR SW may analyze a resistance of the microorganism sample.

The units described herein may be implemented using hardware components and software components. For example, the hardware components may include microphones, amplifiers, band-pass filters, audio to digital convertors, and processing devices. A processing device may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. In particular, the software and data may be stored by one or more computer readable recording mediums.

The methods according to the above-described embodiments may be recorded, stored, or fixed in one or more non-transitory computer-readable media that includes program instructions to be implemented by a computer to cause a processor to execute or perform the program instructions. The media may also include, alone or in combination with the program instructions, data files, data structures, and the like. The program instructions recorded on the media may be those specially designed and constructed, or they may be of the kind well-known and available to those having skill in the computer software arts. Examples of non-transitory computer-readable media include magnetic media such as hard disks, floppy disks, and magnetic tape; optical media such as CD ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations and methods described above, or vice versa.

A number of example embodiments have been described above. Nevertheless, it should be understood that various modifications may be made to these example embodiments. For example, suitable results may be achieved if the described techniques are performed in a different order and/or if components in a described system, architecture, device, or circuit are combined in a different manner and/or replaced or supplemented by other components or their equivalents.

Accordingly, other implementations are within the scope of the following claims.

1. An apparatus for determining a resistance of microorganisms, the apparatus comprising:
a storage including a database to store first region mass spectrum data associated with a plurality of microorganisms and second region mass spectrum data used for determining a resistance of each of the plurality of microorganisms; and
a processor configured to determine a resistance to an antibiotic by inputting mass spectrum data of a microorganism sample to be determined, to a statistic or machine-learning resistance determining module that determines a resistance using the second region mass spectrum data as input data.

2. The apparatus of claim 1, further comprising:
a mass analyzer configured to acquire the first region mass spectrum data and the second region mass spectrum data based on a matrix assisted laser desorption/ionization time-of-flight mass spectrometry (MALDI-TOF MS) in association with the microorganism sample to be determined.

3. The apparatus of claim 2, wherein the processor is configured to identify a microorganism by inputting mass spectrum data of a microorganism sample to be determined, to a statistic or machine-learning identifying module that identifies a microorganism using the first region mass spectrum data as input data.

4. An apparatus for determining a resistance of a microorganism, the apparatus comprising:
a communicator configured to connect to a cloud server including a database that stores first region mass spectrum data associated with a plurality of microorganisms and second region mass spectrum data used for determining a resistance of each of the plurality of microorganisms; and
a processor configured to determine a resistance to an antibiotic by inputting mass spectrum data of a microorganism sample to be determined, to a statistic or machine-learning resistance determining module that determines a resistance using the second region mass spectrum data as input data.

5. The apparatus of claim 4, further comprising:
a mass analyzer configured to acquire the first region mass spectrum data and the second region mass spectrum data based on a matrix assisted laser desorption/ionization time-of-flight mass spectrometry (MALDI-TOF MS) in association with the microorganism sample to be determined.

6. The apparatus of claim 5, wherein the processor is configured to identify a microorganism by inputting mass spectrum data of a microorganism sample to be determined, to a statistic or machine-learning identifying module that identifies a microorganism using the first region mass spectrum data as input data.

7. A method of determining a resistance of a microorganism, the method comprising:
storing, by a storage, first region mass spectrum data associated with a plurality of microorganisms and second region mass spectrum data used for determining a resistance of each of the plurality of microorganisms in a database; and
determining, by a processor, a resistance to an antibiotic by inputting mass spectrum data of a microorganism sample to be determined, to a statistic or machine-learning resistance determining module that determines a resistance using the second region mass spectrum data as input data.

8. The method of claim 7, further comprising:
acquiring, by a mass analyzer, the first region mass spectrum data and the second region mass spectrum data based on a matrix assisted laser desorption/ionization time-of-flight mass spectrometry (MALDI-TOF MS) in association with the microorganism sample to be determined.

9. The method of claim 8, further comprising:
identifying, by the processor, a microorganism by inputting mass spectrum data of a microorganism sample to be determined, to a statistic or machine-learning identifying module that identifies a microorganism using the first region mass spectrum data as input data.

10. An operation method of a microorganism resistance determining apparatus, the method comprising:
acquiring, by a mass analyzer, first region mass spectrum data associated with a microorganism sample to be determined;
identifying, by a processor, a microorganism by inputting the first region mass spectrum data of the microorganism sample to be determined, to a statistic or machine-learning identifying module that identifies a microorganism using mass spectrum data as input data;
acquiring, by the mass analyzer, second region mass spectrum data associated with the microorganism sample; and
determining a resistance to an antibiotic by inputting mass spectrum data of the microorganism sample to a statistic or machine-learning resistance determining module that determines a resistance using the first region and second region mass spectrum data as input data.

11. The operation method of claim 10, further comprising:
storing, by a storage, first region mass spectrum data associated with a plurality of microorganisms and second region mass spectrum data used for determining a resistance of each of the plurality of microorganisms in a database.

12. The operation method of claim 11, wherein the machine-learning resistance determining module is configured to determine whether a microorganism has a resistance to an antibiotic through a machine learning performed by converting the first region mass spectrum data and the second region mass spectrum data into a feature matrix and applying the feature matrix as an input value.

13. The operation method of claim 12, further comprising:
calibrating the microorganism sample using a correction material acting on the second region to increase an accuracy of mass spectrum data on the second region.

14. The operation method of claim 10, further comprising:
interworking, by a communicator, with a cloud server that stores first region mass spectrum data associated with a plurality of microorganisms and second region mass spectrum data used for determining a resistance of each of the plurality of microorganisms, in a database.

15. A non-transitory computer-readable medium comprising a program for instructing a computer to perform the method of claim 8.
